SEQUENCE LISTING (129 sequences)

SEQ ID NO: 1 - DNA, 19 bp, synthetic construct
  source 1..19: mol_type = other DNA
ccctattaaccactcacgg

SEQ ID NO: 2 - DNA, 18 bp, synthetic construct
  source 1..18: mol_type = other DNA
acaaagaaccctaacacc

SEQ ID NO: 3 - DNA, 19 bp, synthetic construct
  source 1..19: mol_type = other DNA
cagggttggtcaatttcgt

SEQ ID NO: 4 - DNA, 19 bp, synthetic construct
  source 1..19: mol_type = other DNA
gtggcaagaaatgggctac

SEQ ID NO: 5 - DNA, 20 bp, synthetic construct
  source 1..20: mol_type = other DNA
accttactaccagacaacct

SEQ ID NO: 6 - DNA, 20 bp, synthetic construct
  source 1..20: mol_type = other DNA
atagtaggcctaaaagcagc

SEQ ID NO: 7 - DNA, 20 bp, synthetic construct
  source 1..20: mol_type = other DNA
cagtgacacatgtttaacgg

SEQ ID NO: 8 - DNA, 20 bp, synthetic construct
  source 1..20: mol_type = other DNA
ctcgatgttggatcaggaca

SEQ ID NO: 9 - DNA, 20 bp, synthetic construct
  source 1..20: mol_type = other DNA
atatacaactacgcaaaggc

SEQ ID NO: 10 - DNA, 20 bp, synthetic construct
  source 1..20: mol_type = other DNA
ctctccacccttatcacaac

SEQ ID NO: 11 - DNA, 20 bp, synthetic construct
  source 1..20: mol_type = other DNA
cccatcctaaagtaaggtca

SEQ ID NO: 12 - DNA, 19 bp, synthetic construct
  source 1..19: mol_type = other DNA
tcaagtatttcctcacgca

SEQ ID NO: 13 - DNA, 20 bp, synthetic construct
  source 1..20: mol_type = other DNA
agttctaccgtacaacccta

SEQ ID NO: 14 - DNA, 20 bp, synthetic construct
  source 1..20: mol_type = other DNA
ctccccatatctaacaacgt

SEQ ID NO: 15 - DNA, 20 bp, synthetic construct
  source 1..20: mol_type = other DNA
ggcttcaatctacttctccc

SEQ ID NO: 16 - DNA, 18 bp, synthetic construct
  source 1..18: mol_type = other DNA
ttatcgtcacagcccatg

SEQ ID NO: 17 - DNA, 20 bp, synthetic construct
  source 1..20: mol_type = other DNA
ctgatccgtcctaatcacag

SEQ ID NO: 18 - DNA, 20 bp, synthetic construct
  source 1..20: mol_type = other DNA
aagtatttagctgactcgcc

SEQ ID NO: 19 - DNA, 20 bp, synthetic construct
  source 1..20: mol_type = other DNA
tgcatacaccacatgaaaca

SEQ ID NO: 20 - DNA, 20 bp, synthetic construct
  source 1..20: mol_type = other DNA
acgctacttcccctatcata

SEQ ID NO: 21 - DNA, 20 bp, synthetic construct
  source 1..20: mol_type = other DNA
ttgacgttgacaatcgagta

SEQ ID NO: 22 - DNA, 19 bp, synthetic construct
  source 1..19: mol_type = other DNA
agattaagagaaccaacac

SEQ ID NO: 23 - DNA, 21 bp, synthetic construct
  source 1..21: mol_type = other DNA
caaactaacctcaaaacaaat

SEQ ID NO: 24 - DNA, 22 bp, synthetic construct
  source 1..22: mol_type = other DNA
accaatagccctggccgtacgc

SEQ ID NO: 25 - DNA, 20 bp, synthetic construct
  source 1..20: mol_type = other DNA
ctaggcctactaaccaacac

SEQ ID NO: 26 - DNA, 20 bp, synthetic construct
  source 1..20: mol_type = other DNA
cctgagctcaccatagtcta

SEQ ID NO: 27 - DNA, 20 bp, synthetic construct
  source 1..20: mol_type = other DNA
tctattgatgagggtcttac

SEQ ID NO: 28 - DNA, 20 bp, synthetic construct
  source 1..20: mol_type = other DNA
tacaaacaactaacctgcca

SEQ ID NO: 29 - DNA, 20 bp, synthetic construct
  source 1..20: mol_type = other DNA
ttgccatactagtctttgcc

SEQ ID NO: 30 - DNA, 21 bp, synthetic construct
  source 1..21: mol_type = other DNA
aacgccacttatccagtgaac

SEQ ID NO: 31 - DNA, 20 bp, synthetic construct
  source 1..20: mol_type = other DNA
tactcttaaaactaggcggc

SEQ ID NO: 32 - DNA, 20 bp, synthetic construct
  source 1..20: mol_type = other DNA
ggacttcaaactctactccc

SEQ ID NO: 33 - DNA, 20 bp, synthetic construct
  source 1..20: mol_type = other DNA
gacatcattaccgggttttc

SEQ ID NO: 34 - DNA, 20 bp, synthetic construct
  source 1..20: mol_type = other DNA
tatcagtctcttccccacaa

SEQ ID NO: 35 - DNA, 20 bp, synthetic construct
  source 1..20: mol_type = other DNA
agccattcaagcaatcctat

SEQ ID NO: 36 - DNA, 20 bp, synthetic construct
  source 1..20: mol_type = other DNA
aaaatcgtagccttctccac

SEQ ID NO: 37 - DNA, 21 bp, synthetic construct
  source 1..21: mol_type = other DNA
ctcgaataattcttctcaccc

SEQ ID NO: 38 - DNA, 20 bp, synthetic construct
  source 1..20: mol_type = other DNA
ctcctagacctaacctgact

SEQ ID NO: 39 - DNA, 20 bp, synthetic construct
  source 1..20: mol_type = other DNA
gccatcgctgtagtatatcc

SEQ ID NO: 40 - DNA, 18 bp, synthetic construct
  source 1..18: mol_type = other DNA
catccaacatctccgcat

SEQ ID NO: 41 - DNA, 20 bp, synthetic construct
  source 1..20: mol_type = other DNA
aaacttactatccgccatcc

SEQ ID NO: 42 - DNA, 20 bp, synthetic construct
  source 1..20: mol_type = other DNA
caagcccgaatgatatttcc

SEQ ID NO: 43 - DNA, 20 bp, synthetic construct
  source 1..20: mol_type = other DNA
ttaactccaccattagcacc

SEQ ID NO: 44 - DNA, 20 bp, synthetic construct
  source 1..20: mol_type = other DNA
aaagccatttaccgtacata

SEQ ID NO: 45 - DNA, 19 bp, synthetic construct
  source 1..19: mol_type = other DNA
agtgctgtggccagaagcg

SEQ ID NO: 46 - DNA, 19 bp, synthetic construct
  source 1..19: mol_type = other DNA
cattgctgcgtgcttgatg

SEQ ID NO: 47 - DNA, 19 bp, synthetic construct
  source 1..19: mol_type = other DNA
gctgagcaagaggtggtga

SEQ ID NO: 48 - DNA, 20 bp, synthetic construct
  source 1..20: mol_type = other DNA
gtttagctcagagcggtcaa

SEQ ID NO: 49 - DNA, 20 bp, synthetic construct
  source 1..20: mol_type = other DNA
gtgtccaaagagctgttcct

SEQ ID NO: 50 - DNA, 20 bp, synthetic construct
  source 1..20: mol_type = other DNA
atactggtgatgctagaggt

SEQ ID NO: 51 - DNA, 20 bp, synthetic construct
  source 1..20: mol_type = other DNA
tagggtaacttgttccgttg

SEQ ID NO: 52 - DNA, 20 bp, synthetic construct
  source 1..20: mol_type = other DNA
cgttcggtaagcattaggaa

SEQ ID NO: 53 - DNA, 20 bp, synthetic construct
  source 1..20: mol_type = other DNA
tagaatgatggctagggtga

SEQ ID NO: 54 - DNA, 20 bp, synthetic construct
  source 1..20: mol_type = other DNA
taggaggtgtatgagttggt

SEQ ID NO: 55 - DNA, 21 bp, synthetic construct
  source 1..21: mol_type = other DNA
agcatgtttatttctaggcct

SEQ ID NO: 56 - DNA, 20 bp, synthetic construct
  source 1..20: mol_type = other DNA
tgggtaattgaggagtatgc

SEQ ID NO: 57 - DNA, 20 bp, synthetic construct
  source 1..20: mol_type = other DNA
tgtgattgaggtggagtaga

SEQ ID NO: 58 - DNA, 20 bp, synthetic construct
  source 1..20: mol_type = other DNA
gattagggtgcttagctgtt

SEQ ID NO: 59 - DNA, 20 bp, synthetic construct
  source 1..20: mol_type = other DNA
cgttgtagatgtggtcgtta

SEQ ID NO: 60 - DNA, 20 bp, synthetic construct
  source 1..20: mol_type = other DNA
gcgtttggtattgggttatg

SEQ ID NO: 61 - DNA, 20 bp, synthetic construct
  source 1..20: mol_type = other DNA
gatgattatggtagcggagg

SEQ ID NO: 62 - DNA, 20 bp, synthetic construct
  source 1..20: mol_type = other DNA
agtgttgtgggaagaaagtt

SEQ ID NO: 63 - DNA, 18 bp, synthetic construct
  source 1..18: mol_type = other DNA
tagacctacttgcgctgc

SEQ ID NO: 64 - DNA, 19 bp, synthetic construct
  source 1..19: mol_type = other DNA
ggggaagtatgtaggagtt

SEQ ID NO: 65 - DNA, 20 bp, synthetic construct
  source 1..20: mol_type = other DNA
ctttacagtgggctctagag

SEQ ID NO: 66 - DNA, 20 bp, synthetic construct
  source 1..20: mol_type = other DNA
attagtcattgttgggtggt

SEQ ID NO: 67 - DNA, 20 bp, synthetic construct
  source 1..20: mol_type = other DNA
atgagtaggctgatggtttc

SEQ ID NO: 68 - DNA, 21 bp, synthetic construct
  source 1..21: mol_type = other DNA
gcgttatggagtggaagtgaa

SEQ ID NO: 69 - DNA, 20 bp, synthetic construct
  source 1..20: mol_type = other DNA
tgattgatactcctgatgcg

SEQ ID NO: 70 - DNA, 20 bp, synthetic construct
  source 1..20: mol_type = other DNA
accacatctacaaaatgcca

SEQ ID NO: 71 - DNA, 20 bp, synthetic construct
  source 1..20: mol_type = other DNA
tcatggtaggggtaaaagga

SEQ ID NO: 72 - DNA, 20 bp, synthetic construct
  source 1..20: mol_type = other DNA
ggtgttgagggttatgagag

SEQ ID NO: 73 - DNA, 20 bp, synthetic construct
  source 1..20: mol_type = other DNA
gggtaggagtcaggtagtta

SEQ ID NO: 74 - DNA, 20 bp, synthetic construct
  source 1..20: mol_type = other DNA
cgacatgggctttagggagt

SEQ ID NO: 75 - DNA, 20 bp, synthetic construct
  source 1..20: mol_type = other DNA
gaggattatgatgcgactgt

SEQ ID NO: 76 - DNA, 19 bp, synthetic construct
  source 1..19: mol_type = other DNA
tgagggataggaggagaat

SEQ ID NO: 77 - DNA, 20 bp, synthetic construct
  source 1..20: mol_type = other DNA
ataaaggtggatgcgacaat

SEQ ID NO: 78 - DNA, 20 bp, synthetic construct
  source 1..20: mol_type = other DNA
caactgatgagcaagaagga

SEQ ID NO: 79 - DNA, 20 bp, synthetic construct
  source 1..20: mol_type = other DNA
atgtcattttgtgtaagggc

SEQ ID NO: 80 - DNA, 19 bp, synthetic construct
  source 1..19: mol_type = other DNA
ggaggtagcgatgagagta

SEQ ID NO: 81 - DNA, 19 bp, synthetic construct
  source 1..19: mol_type = other DNA
ctagataggggattgtgcg

SEQ ID NO: 82 - DNA, 20 bp, synthetic construct
  source 1..20: mol_type = other DNA
ttagcgatggaggtaggatt

SEQ ID NO: 83 - DNA, 20 bp, synthetic construct
  source 1..20: mol_type = other DNA
ggtcgatgaatgagtggtta

SEQ ID NO: 84 - DNA, 20 bp, synthetic construct
  source 1..20: mol_type = other DNA
gaatgatatttggcctcacg

SEQ ID NO: 85 - DNA, 20 bp, synthetic construct
  source 1..20: mol_type = other DNA
gtgtttaaggggttggctag

SEQ ID NO: 86 - DNA, 20 bp, synthetic construct
  source 1..20: mol_type = other DNA
aaggttttcatctccggttt

SEQ ID NO: 87 - DNA, 20 bp, synthetic construct
  source 1..20: mol_type = other DNA
gtgggtaggtttgttggtat

SEQ ID NO: 88 - DNA, 20 bp, synthetic construct
  source 1..20: mol_type = other DNA
tgatagacctgtgatccatc

SEQ ID NO: 89 - DNA, 30 bp, synthetic construct
  source 1..30: mol_type = other DNA
ccatctcatccctgcgtgtctccgactcag

SEQ ID NO: 90 - DNA, 23 bp, synthetic construct
  source 1..23: mol_type = other DNA
cctctctatgggcagtcggtgat

SEQ ID NO: 91 - DNA, 10 bp, synthetic construct
  source 1..10: mol_type = other DNA
ctaaggtagc

SEQ ID NO: 92 - DNA, 10 bp, synthetic construct
  source 1..10: mol_type = other DNA
aagaggaatc

SEQ ID NO: 93 - DNA, 10 bp, synthetic construct
  source 1..10: mol_type = other DNA
cagatggaac

SEQ ID NO: 94 - DNA, 10 bp, synthetic construct
  source 1..10: mol_type = other DNA
ttggtgattc

SEQ ID NO: 95 - DNA, 10 bp, synthetic construct
  source 1..10: mol_type = other DNA
ttccgagaat

SEQ ID NO: 96 - DNA, 10 bp, synthetic construct
  source 1..10: mol_type = other DNA
tgagtggatc

SEQ ID NO: 97 - DNA, 10 bp, synthetic construct
  source 1..10: mol_type = other DNA
tacgtggtac

SEQ ID NO: 98 - DNA, 10 bp, synthetic construct
  source 1..10: mol_type = other DNA
tctatcggat

SEQ ID NO: 99 - DNA, 10 bp, synthetic construct
  source 1..10: mol_type = other DNA
ttggagtctc

SEQ ID NO: 100 - DNA, 10 bp, synthetic construct
  source 1..10: mol_type = other DNA
tctggatgac

SEQ ID NO: 101 - DNA, 10 bp, synthetic construct
  source 1..10: mol_type = other DNA
cagatccatc

SEQ ID NO: 102 - DNA, 10 bp, synthetic construct
  source 1..10: mol_type = other DNA
ttcgagacgc

SEQ ID NO: 103 - DNA, 10 bp, synthetic construct
  source 1..10: mol_type = other DNA
tgccacgaac

SEQ ID NO: 104 - DNA, 10 bp, synthetic construct
  source 1..10: mol_type = other DNA
aacctcattc

SEQ ID NO: 105 - DNA, 10 bp, synthetic construct
  source 1..10: mol_type = other DNA
cctgagatac

SEQ ID NO: 106 - DNA, 10 bp, synthetic construct
  source 1..10: mol_type = other DNA
ttacaacctc

SEQ ID NO: 107 - DNA, 10 bp, synthetic construct
  source 1..10: mol_type = other DNA
aaccatccgc

SEQ ID NO: 108 - DNA, 10 bp, synthetic construct
  source 1..10: mol_type = other DNA
atccggaatc

SEQ ID NO: 109 - DNA, 10 bp, synthetic construct
  source 1..10: mol_type = other DNA
tcgaccactc

SEQ ID NO: 110 - DNA, 10 bp, synthetic construct
  source 1..10: mol_type = other DNA
cgaggttatc

SEQ ID NO: 111 - DNA, 10 bp, synthetic construct
  source 1..10: mol_type = other DNA
tcttacacac

SEQ ID NO: 112 - DNA, 12 bp, synthetic construct
  source 1..12: mol_type = other DNA
ttctcattgaac

SEQ ID NO: 113 - DNA, 11 bp, synthetic construct
  source 1..11: mol_type = other DNA
tcgcatcgttc

SEQ ID NO: 114 - DNA, 12 bp, synthetic construct
  source 1..12: mol_type = other DNA
taagccattgtc

SEQ ID NO: 115 - DNA, 11 bp, synthetic construct
  source 1..11: mol_type = other DNA
aaggaatcgtc

SEQ ID NO: 116 - DNA, 12 bp, synthetic construct
  source 1..12: mol_type = other DNA
cttgagaatgtc

SEQ ID NO: 117 - DNA, 12 bp, synthetic construct
  source 1..12: mol_type = other DNA
tggaggacggac

SEQ ID NO: 118 - DNA, 11 bp, synthetic construct
  source 1..11: mol_type = other DNA
taacaatcggc

SEQ ID NO: 119 - DNA, 11 bp, synthetic construct
  source 1..11: mol_type = other DNA
ctgacataatc

SEQ ID NO: 120 - DNA, 11 bp, synthetic construct
  source 1..11: mol_type = other DNA
ttccacttcgc

SEQ ID NO: 121 - DNA, 10 bp, synthetic construct
  source 1..10: mol_type = other DNA
agcacgaatc

SEQ ID NO: 122 - DNA, 11 bp, synthetic construct
  source 1..11: mol_type = other DNA
cttgacaccgc

SEQ ID NO: 123 - DNA, 12 bp, synthetic construct
  source 1..12: mol_type = other DNA
ttggaggccagc

SEQ ID NO: 124 - DNA, 12 bp, synthetic construct
  source 1..12: mol_type = other DNA
tggagcttcctc

SEQ ID NO: 125 - DNA, 11 bp, synthetic construct
  source 1..11: mol_type = other DNA
tcagtccgaac

SEQ ID NO: 126 - DNA, 12 bp, synthetic construct
  source 1..12: mol_type = other DNA
taaggcaaccac

SEQ ID NO: 127 - DNA, 11 bp, synthetic construct
  source 1..11: mol_type = other DNA
ttctaagagac

SEQ ID NO: 128 - DNA, 12 bp, synthetic construct
  source 1..12: mol_type = other DNA
tcctaacataac

SEQ ID NO: 129 - DNA, 11 bp, synthetic construct
  source 1..11: mol_type = other DNA
cggacaatggc

(19) United States
(12) Patent Application Publication
(10) Pub. No.: US 2024/0102087 A1
(43) Pub. Date: Mar. 28, 2024

(54) KIT FOR HIGH-THROUGHPUT SEQUENCING (HTS) OF HUMAN MITOCHONDRIAL GENOME BY DIRECT AMPLIFICATION WITH FUSION PRIMER

(71) Applicant: IPE BIOTECHNOLOGY CO., LTD., Beijing (CN)

(72) Inventors: Yajiao Pan, Beijing (CN); Baowang Qu, Beijing (CN); Chaohui Chen, Beijing (CN)

(21) Appl. No.: 18/003,243

(22) PCT Filed: Jul. 13, 2022
(86) PCT No.: PCT/CN2022/105384

§ 371 Date: Dec. 23, 2022

(30) Foreign Application Priority Data
Jul. 13, 2021 (CN) 202110788220.5

(51) Int. Cl. C12Q 1/6855; C12Q 1/6869

(52) U.S. Cl. CPC C12Q 1/6855; C12Q 1/6869

(57) ABSTRACT

The present disclosure provides a high-throughput sequencing (HTS) kit of a human mitochondrial whole genome based on direct amplification of a fusion primer, including a library preparation kit, a sequencing template preparation kit, and a sequencing kit; where the library preparation kit includes a multiplex PCR primer pool tagged with different sample tags, a DNA extraction-free PCR amplification enzyme, a PCR reaction buffer, a 2800 control DNA, and a DNA purification magnetic bead. In the present disclosure, the kit for HTS of human mitochondrial genome by direct amplification with fusion primer has a low detection cost and a convenient operation.

### CROSS REFERENCE TO RELATED APPLICATION

The present application is a U.S. National Phase application of PCT International Application Number PCT/CN2022/105384, filed on Jul. 13, 2022, which claims priority to Chinese Patent Application No. 202110788220.5, filed with the China National Intellectual Property Administration (CNIPA) on Jul. 13, 2021 and entitled “KIT FOR HIGH-THROUGHPUT SEQUENCING (HTS) OF HUMAN MITOCHONDRIAL GENOME BY DIRECT AMPLIFICATION WITH FUSION PRIMER”, which is incorporated herein by reference in its entirety.

### REFERENCE TO SEQUENCE LISTING

A computer readable XML file entitled “SeqList-BGI016.001APC”, that was created on Dec. 22, 2022, with a file size of about 114,227 bytes, contains the sequence listing for this application, has been filed with this application, and is hereby incorporated by reference in its entirety.

### TECHNICAL FIELD

The present disclosure relates to the technical fields of forensic medicine, criminal investigation, and material evidence identification, in particular to a kit for high-throughput sequencing (HTS) of human mitochondrial genome by direct amplification with fusion primer.

### BACKGROUND

Mitochondrial DNA plays an important role in the field of human origin and evolution researches and forensic identification. Mitochondrial DNA has the genetic characteristics of maternal genetic non recombination, which can be used for the human origin and evolution researches. Compared with nuclear DNA, the mitochondrial DNA has a high copy number and a stable structure, and can be preserved intact under harsh conditions, which is suitable for the identification of old and highly-degraded samples in forensics.

The human mitochondrial genome sequence is a circular genetic material with a length of 16,569 bp, and can be divided into a control region of 1,122 bp and a coding region of 15,447 bp according to functions, where the control region includes 3 hypervariable regions with a desirable polymorphism.

Currently, researches on mitochondria are generally limited to single nucleotide polymorphisms (SNPs) of the hypervariable regions of the control region and some characteristic coding regions. However, testing only a subset of mitochondrial regions may reduce the polymorphic information content, leading to an increase in the number of random matches and a reduction in the system's ability to exclude, thereby increasing the likelihood of wrongful convictions.

There is no doubt that mitochondrial genome-wide data can provide more accurate information than current data. Most of the current mitochondrial genome data is obtained by traditional Sanger sequencing, which is time-consuming and labor-intensive. High-throughput sequencing (HTS), also known as massive parallel sequencing (MPS), can sequence hundreds of thousands to millions of DNA molecules from multiple samples at one time, providing a new technical means for DNA analysis. However, the MPS requires operations such as DNA extraction and ligation, with complicated processes and poor work efficiency.

### SUMMARY

The present disclosure aims to provide a kit for HTS of human mitochondrial genome by direct amplification with fusion primer. The kit has a low detection cost and a convenient operation.

The present disclosure provides a kit for HTS of human mitochondrial genome by direct amplification with fusion primer, including a library preparation kit, a sequencing template preparation kit, and a sequencing kit; where
the library preparation kit includes a multiplex PCR primer pool tagged with different sample tags, a DNA extraction-free PCR amplification enzyme, a PCR reaction buffer, a 2800 control DNA, and a DNA purification magnetic bead.

Preferably, the multiplex PCR primer pool includes sample tag sequences shown in SEQ ID NO: 91 to SEQ ID NO: 129 and tcacgaata.

Preferably, the sequencing template preparation kit is purchased from Thermo Fisher Scientific.

Preferably, the sequencing kit is purchased from Thermo Fisher Scientific.

A preparation method of the kit for HTS of human mitochondrial genome by direct amplification with fusion primer includes the following steps:
(1) designing a fusion primer including a specific primer, a sample tag, and an adapter sequence;
(2) establishing a DNA extraction-free PCR system: the PCR system includes a special amplification enzyme that resists PCR inhibitory components in blood and a corresponding buffer;
(3) establishing a locus library that is free from DNA extraction and directly amplified by the fusion primer;
(4) conducting an emulsion DNA polymerase chain reaction (ePCR) to obtain a sequencing template, and forming an independent PCR micro-reaction pool by emulsion coverage of particles carrying a single DNA fragment, to achieve independent parallel amplification of an entire fragment library;
(5) conducting high-throughput DNA sequencing; and
(6) conducting a data analysis and displaying reported results.

Therefore, in the present disclosure, the kit for HTS of human mitochondrial genome by direct amplification with fusion primer is used to form a complete set of kits that are suitable for a high-throughput DNA sequencing platform to achieve parallel and stable testing of a multi-sample mitochondrial whole genome.

The kit is free of DNA extraction or ligation library construction, and can complete a single assay within one working day; in addition, the kit can also detect the mitochondrial whole genomes from dozens to hundreds of people in one assay, with its assay cost and operation time allowing large-scale library construction.

In the present disclosure, the high-throughput DNA sequencing-based detection kit of the human mitochondrial whole genome includes all reagents for library preparation, water-in-oil PCR sequencing template preparation, and high-throughput sequencing procedures, and has the following technical effects:
(1) detection of the mitochondrial whole genome sequence can improve the content of polymorphism information;
(2) the parallel testing of multiple samples is realized to improve a detection efficiency; and
(3) DNA extraction-free and ligation-free library construction by direct amplification achieves a library construction time reduced to 2 h and a single assay time reduced to one day.

The technical solutions of the present disclosure will be further described in detail below with reference to drawings and examples.

### BRIEF DESCRIPTION OF THE DRAWINGS

FIG. 1 shows a preparation technology flow chart of the kit for HTS of human mitochondrial genome by direct amplification with fusion primer of the present disclosure;

FIG. 2 shows an electrophoresis schematic diagram of a multiplex PCR amplification efficiency of different template types under a DNA extraction-free PCR system (10 mL); and

FIG. 3 shows a schematic diagram of sequencing results of a mitochondrial whole genome sequence with an Ion S5XLTM sequencing platform.

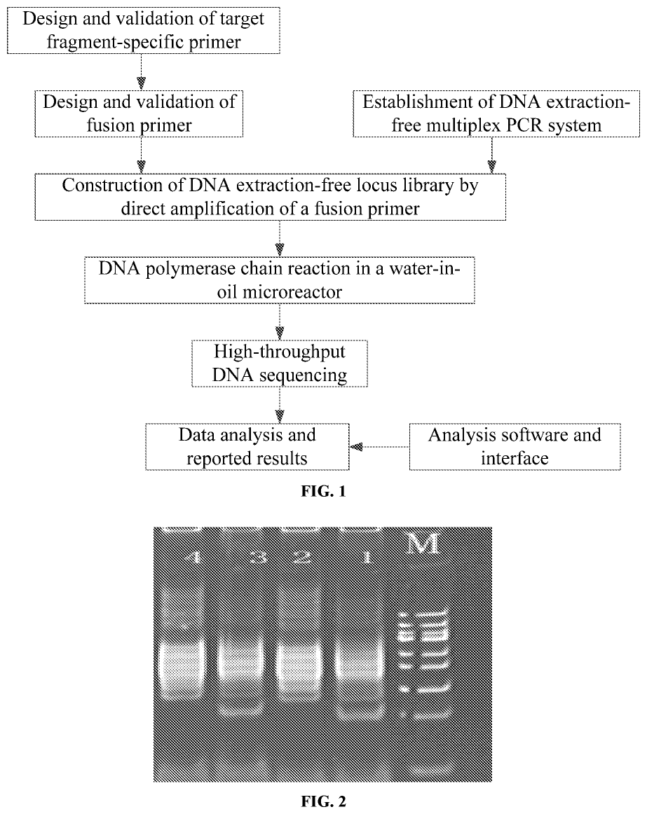

### DETAILED DESCRIPTION OF THE EMBODIMENTS

The technical solutions of the present disclosure will be further described below with reference to the accompanying drawings and examples.

### Example 1

Construction of a Sequencing Library by Direct Amplification

Propagation sub-library refers to DNA fragments ligated with different adapters at both ends, where one side is a sequencing adapter containing sample tags to distinguish the sequencing results of different samples; and the other side is a fixating adapter for ligating captured particles.

The propagation sub-library has the following structure: adapter P—target amplicon—sample tag—adapter A universal part.

There was a fusion primer including a target fragment-specific primer, adapters, and a sample tag, and a PCR amplification enzyme and a buffer solution with blood-derived amplification capabilities; a DNA library composed of multiple STR target fragments was directly obtained by multiplex PCR amplification of blood samples, and the DNA library had two ends ligated to different adapter sequences, and had sample tags. This omitted several steps of DNA extraction, monoplex PCR, PCR product mixing, and adapter ligation for existing high-throughput DNA sequencing library construction.

TABLE 1
Composition of PCR system for constructing sequencing library by direct amplification

| Component | Function | Ways to obtain |
|---|---|---|
| Fusion | The fusion primer attaches adapters and sample | Fusion primer design, including |
| primer | tags to amplification products for subsequent | design of target fragment-specific |
| | high-throughput sequencing reactions and | primers, adapters, and sample tags |
| | sample differentiation. | |
| PCR | The PCR amplification enzyme is an anti-blood | Screening of mutated Taq |
| amplification | PCR inhibitor, such that blood can be used | engineered bacteria |
| enzyme | directly as a PCR template to avoid DNA | |
| | extraction. | |
| PCR | The PCR buffer enhances a PCR amplification | Preparation of PCR buffers, |
| buffer | efficiency, such that blood can be used directly | including PCR enhancers and |
| | as a PCR template. | pH adjustment |
| Fusion | The ratio of each fusion primer is adjusted such | Multiplex PCR balance |
| primer | that the amount of PCR product of each STR | adjustment |
| ratio | locus is basically the same. | |

I. Design of Fusion Primer

In addition to a target fragment-specific primer, the fusion primer further had other long primer sequences including sequencing adapters, fixating adapters, and sample tags. The fusion primer had the following structure, where the adapter A was a sequencing primer region, the adapter P was a captured particle binding region, and the sample tag was used to distinguish different samples.

Upstream Fusion Primer:

5′-Adapter A (30 Bases)-Sample Tag (10 Bases)-Target-Fragment Upstream Primer (19 to 23 Bases)-3′

Downstream Fusion Primer:

5′-Adapter P (23 Bases)-Target-Fragment Downstream Primer (19 to 23 Bases)-3′

The target fragment-specific primer was used to amplify a target fragment of the human mitochondrial whole genome; the adapter sequences included a fixating adapter and a sequencing adapter, which were used to bind captured magnetic beads and a sequencing primer, respectively, so as to complete subsequent water-in-oil PCR and sequencing reactions. The sample tag sequence was used to distinguish different samples.

(1) Design and Verification of Primers for Target Fragment of Human Mitochondrial Whole Genome

Based on the NC_012920.1 Cambridge revised version of the human mitochondrial whole genome sequence, primers for the target fragment were designed using a Primer5.0 online tool. An NC_012920.1 reference genome was 16,569 bp. Table 2 showed the primer design distribution. SEQ ID NO: 1 to SEQ ID NO: 44 were upstream primers, and SEQ ID NO: 45 to SEQ ID NO: 88 were corresponding downstream primers. The primers included an end and a beginning of a mitochondrial reference genome (the mitochondria were circular). The specificity and content of an amplified product were detected by agarose electrophoresis, and the accuracy of an amplified product sequence was detected by a sequencing method to prove its usability.

TABLE 2
Sequences of specific primers corresponding
to mitochondrial genome positions

| Sequence | Mitochondrial |
|---|---|
| No: | genome location |
| 1, 45 | 17-332 |
| 2, 46 | 361-774 |
| 3, 47 | 869-1253 |
| 4, 48 | 1347-1677 |
| 5, 49 | 1697-2125 |
| 6, 50 | 2167-2531 |
| 7, 51 | 2549-2932 |
| 8, 52 | 2986-3380 |
| 9, 53 | 3395-3756 |
| 10, 54 | 3790-4173 |
| 11, 55 | 4187-4581 |
| 12, 56 | 4639-5026 |
| 13, 57 | 5045-5378 |
| 14, 58 | 5382-5713 |
| 15, 59 | 5718-6069 |
| 16, 60 | 6070-6447 |
| 17, 61 | 6458-6839 |
| 18, 62 | 6859-7191 |
| 19, 63 | 7238-7612 |
| 20, 64 | 7617-7958 |
| 21, 65 | 7992-8304 |
| 22, 66 | 8333-8675 |
| 23, 67 | 8676-8978 |
| 24, 68 | 8982-9330 |
| 25, 69 | 9339-9639 |
| 26, 70 | 9640-9949 |
| 27, 71 | 9973-10289 |
| 28, 72 | 10294-10622 |
| 29, 73 | 10651-10981 |
| 30, 74 | 11002-11423 |
| 31, 75 | 11460-11793 |
| 32, 76 | 11798-12107 |
| 33, 77 | 12113-12470 |
| 34, 78 | 12471-12806 |
| 35, 79 | 12836-13229 |
| 36, 80 | 13234-13587 |
| 37, 81 | 13607-13957 |
| 38, 82 | 13990-14388 |
| 39, 83 | 14453-14807 |
| 40, 84 | 14817-15166 |
| 41, 85 | 15187-15537 |
| 42, 86 | 15550-15941 |
| 43, 87 | 15971-16294 |
| 44, 88 | 16316-16 |

(2) Design of Adapter Sequences (with a High-Throughput Sequencing Platform)

Different high-throughput sequencing platforms each have specific adapter sequences, with the following characteristics: (1) based on a principle of ion semiconductor sequencing, there is a low sequencing cost; (2) rapidness, only 2 h to 3 h is required for on-machine sequencing; (3) a sequencing read length is 200 to 400 bases, meeting the needs of sequencing read length; (4) strong flexibility, with a variety of chips meeting different throughput requirements.

In the present disclosure, a Thermo Scientific sequencing system was selected as a detection platform, and the corresponding adapter sequences were shown in Table 3. The adapter P was a fixating adapter for binding DNA capturing magnetic beads; the adapter A, as a sequencing adapter, was used for sequencing with a universal primer.

TABLE 3
Adapter sequences

| Sequence | | |
|---|---|---|
| No: | Adapter | Sequence |
| 89 | Adapter A | CCATCTCATCCCTGCGTGTCTCC |
| | | GACTCAG |
| 90 | Adapter P | CCTCTCTATGGGCAGTCGGTGAT |

(3) Design and Verification of Sample Tag Sequences

Samples tested in parallel were distinguished by a unique sample tag; under the premise of sufficient throughput, the number of samples for parallel test was determined by the number of available sample tags. Table 4 listed 40 sample tags validated by the present disclosure.

TABLE 4

| Sample | Sequence |
|---|---|
| tag No. | No: |
| code001 | 91 |
| code002 | 92 |
| code003 | 93 |
| code004 | 94 |
| code005 | 95 |
| code006 | 96 |
| code007 | tcacgaata |
| code008 | 97 |
| code009 | 98 |
| code010 | 99 |
| code011 | 100 |
| code012 | 101 |
| code013 | 102 |
| code014 | 103 |
| code015 | 104 |
| code016 | 105 |
| code017 | 106 |
| code018 | 107 |
| code019 | 108 |
| code020 | 109 |
| code021 | 110 |
| code022 | 111 |
| code023 | 112 |
| code024 | 113 |
| code025 | 114 |
| code026 | 115 |
| code027 | 116 |
| code028 | 117 |
| code029 | 118 |
| code030 | 119 |
| code031 | 120 |
| code032 | 121 |
| code033 | 122 |
| code034 | 123 |
| code035 | 124 |
| code036 | 125 |
| code037 | 126 |
| code038 | 127 |
| code039 | 128 |
| code040 | 129 |

II. Establishment of PCR Amplification System (Including PCR Enzyme and Buffer) for Blood-Derived Direct Amplification

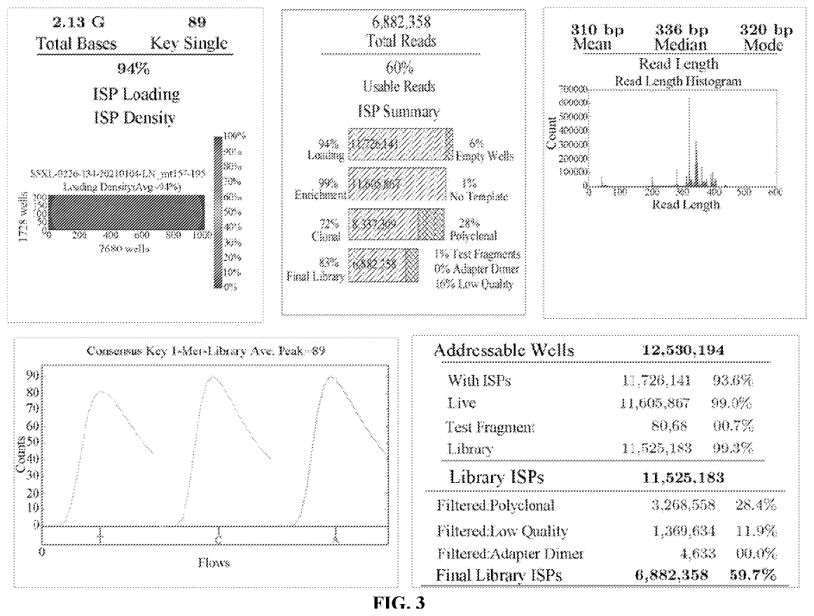

A genetically mutated Taq engineered bacterium was selected. Due to the deletion mutation of about 10 amino acids in an N-terminus, a Taq enzyme protein product of the engineered bacterium had a function of resisting blood PCR inhibitory components. The engineered bacterium was mixed with a PCR buffer that had a higher pH value and contained (NH₄)₂SO₄ and other PCR enhancers. The performance verification results showed that the PCR reaction system could still maintain an ideal multiplex PCR amplification efficiency in the case of containing anti-blood-derived PCR inhibitory components. In FIG. 2, Lanes 1 and 2 were 10 ng genomic DNA templates, Lanes 3 and 4 were blood slice templates with a diameter of 1 mm, and M was a 100 bp marker.

III. Establishment of Balanced Multiplex PCR System

The balance was achieved on an amount of each STR target amplification product by adjusting a ratio of each primer pair in the multiplex PCR amplification system. The Concentrations of 44 pairs of fusion primers in each primer pool of the multiplex PCR system were shown in Table 5.

TABLE 5
Concentrations of 44 pairs of fusion primers in each primer pool

| Primer name (indicated by a | Concentration |
|---|---|
| position of a product in a | (M/PCR reaction |
| mitochondrial genome) | system) |
| 17-332 | 0.01-0.03 |
| 361-774 | 0.05-0.07 |
| 869-1253 | 0.03-0.05 |
| 1347-1677 | 0.03-0.05 |
| 1697-2125 | 0.06-0.08 |
| 2167-2531 | 0.03-0.05 |
| 2549-2932 | 0.03-0.05 |
| 2986-3380 | 0.06-0.08 |
| 3395-3756 | 0.1-0.3 |
| 3790-4173 | 0.03-0.06 |
| 4187-4581 | 0.01-0.04 |
| 4639-5026 | 0.03-0.05 |
| 5045-5378 | 0.05-0.09 |
| 5382-5713 | 0.05-0.08 |
| 5718-6069 | 0.04-0.07 |
| 6070-6447 | 0.04-0.07 |
| 6458-6839 | 0.04-0.07 |
| 6859-7191 | 0.03-0.05 |
| 7238-7612 | 0.05-0.08 |
| 7617-7958 | 0.04-0.07 |
| 7992-8304 | 0.04-0.07 |
| 8333-8675 | 0.04-0.07 |
| 8676-8978 | 0.06-0.08 |
| 8982-9330 | 0.04-0.07 |
| 9339-9639 | 0.03-0.05 |
| 9640-9949 | 0.04-0.07 |
| 9973-10289 | 0.04-0.07 |
| 10294-10622 | 0.03-0.05 |
| 10651-10981 | 0.04-0.07 |
| 11002-11423 | 0.04-0.07 |
| 11460-11793 | 0.1-0.3 |
| 11798-12107 | 0.05-0.08 |
| 12113-12470 | 0.050-0.08 |
| 12471-12806 | 0.05-0.08 |
| 12836-13229 | 0.08-0.12 |
| 13234-13587 | 0.060-0.08 |
| 13607-13957 | 0.1-0.1 |
| 13990-14388 | 0.08-0.12 |
| 14453-14807 | 0.08-0.12 |
| 14817-15166 | 0.04-0.07 |
| 15187-15537 | 0.04-0.07 |
| 15550-15941 | 0.08-0.12 |
| 15971-16294 | 0.04-0.07 |
| 16316-16 | 0.04-0.07 |

### Example 2

DNA Polymerase Chain Reaction (emPCR) in a Water-In-Oil Microreactor to Obtain Sequencing Templates
(1) The library content generated by the multiplex PCR direct amplification was immobilized on the capturing magnetic beads via the adapter P, such that each magnetic bead carried a single DNA fragment.
(2) Emulsification was conducted on a water-phase PCR reagent and an oil-phase reagent to form an emulsion, and the magnetic bead carrying the template was mixed with the emulsion and entered into droplets, where each droplet was a water-in-oil micro-reaction cell.
(3) The amplification of the entire fragment library was conducted in parallel in each water-in-oil micro-reaction cell to form a sequencing template.

### Example 3

Parallel Batched DNA Sequencing

The sequencing was conducted using a high-throughput DNA sequencer, such as Thermo Fisher's PGM or S5/S5 XL, and a sequencing reaction was conducted in a manner of sequencing-by-synthesis.
(1) An emPCR product of the library content was combined with a sequencing universal primer through the adapter A;
(2) 4 kinds of deoxyribonucleoside triphosphates (dNTP, N was A, G, C, and T) were successively involved in the PCR synthesis system;
(3) a DNA polymerization reaction was carried out when the added dNTP was paired with the sequencing template;
(4) a pH change caused by H ions released by the DNA polymerization was recognized, and the sequencing of one base was completed; and
(5) steps 2 to 4 were repeated until the sequencing of the entire DNA fragment was completed.

### Example 4

Data Analysis and Reported Results
(1) Data quality control: the raw data was filtered according to the sequencing length and mass;
(2) classification of sequencing information: according to the sample tag sequences in the sequencing results, the sequencing results were effectively classified into different sample folders; and
(3) according to the alignment with a standard reference sequence, the variation of the mitochondrial genome sequence of the sample was found.

### Example 5

Parallel Detection of Mitochondrial Whole Genome Sequences from 40 Samples

(I) Experimental Materials

Reagents: an HTS kit of a human mitochondrial whole genome (mtDNA) by direct amplification of a fusion primer included:
1) a library preparation kit included 40 sets of multiplex PCR primer pools tagged with different sample tags, a DNA extraction-free PCR amplification enzyme, a PCR reaction buffer, a 2800 control DNA, and a DNA purification magnetic bead;
2) a sequencing template preparation kit (purchased from Thermo Fisher Scientific, USA); and
3) a sequencing kit (purchased from Thermo Fisher Scientific, USA).

Sample: whole blood spotted on a filter paper matrix was used as a sample.

(II) Experimental Procedures

1. Library Preparation

1) Preparation of Samples

With a puncher in a diameter of 1 mm, 39 blood slices were punched into 96-well PCR plates in a certain order, where one blood slice was taken from each sample, and 1 ng of the 2800 control DNA was added to the first well of each 96-well plate.

2) Multiplex PCR

The 40 multiplex PCR systems stored in the 96-well plates each were added to a corresponding PCR plate containing the blood slices at 10 mL per well. 10 mL of the multiplex PCR system included the following components:

| Component name | Component | Volume (mL) |
|---|---|---|
| 5*PCR buffer | PCR enhancers such as | 2.0 |
| | Tris-HCl, Mg²⁺, and (NH₄)₂SO₄ | |
| Taq | PCR amplification enzyme | 0.8 |
| 10 mM dNTPs | 4 kinds of dNTPs | 0.2 |
| PCR Mix1-40 | upstream fusion primers and | 1.0 |
| | downstream fusion primers | |
| | that were tagged with | |
| | different sample tags | |
| ddH₂O | Ultrapure water | 2.0 |

| | |
|---|---|
| Total volume | 10 |

The PCR was conducted following the program below:

| | | |
|---|---|---|
| Temperature | Time | Number of cycles |

| | | | |
|---|---|---|---|
| 95° C. | 3 | m | 1 |
| 95° C. | 30 | s | |
| 60° C. | 30 | s | 22 |
| 68° C. | 1 | m | |
| 68° C. | 10 | m | 1 |

| | | |
|---|---|---|
| 4° C. | hold | hold |

3) Purification of PCR Products
a. 5 μL of the PCR products in each well were mixed, added into a 1.5 mL EP tube, mixed well by shaking, and 50 μL of a mixture was collected for purification.
b. 50 μL of the PCR mixed product was pipetted into a 1.5 mL EP tube, added with 60 mL of purified magnetic beads (the magnetic beads were equilibrated to a room temperature in advance), and the pipette was adjusted to 150 μL for pipetting and beating 10 times to mix well.
c. The mixed solution in step a was equilibrated at a room temperature for 5 min to achieve the optimal recovery effect.
d. The mixture was allowed to stand on a magnetic grate for 10 min.
e. The supernatant was discarded, and the centrifuge tube was removed from the magnetic grate.
f. 200 μL of 70% ethanol was pipetted into a centrifuge tube, pipetted and beat 10 times to fully wash the magnetic beads; the centrifuge tube was placed on the magnetic grate and allowed to stand for 2 min, and the supernatant was discarded.
g. Step e was repeated once.
h. The magnetic beads were fully dried by allowing to stand on the magnetic grate for 10 min.
i. The EP tube was removed from the magnetic grate, washed with 50 μL of nuclease-free water, mixed well by pipetting, and allowed to stand at a room temperature for 30 min, during which time, the EP tube was mixed well 2 to 3 times by pipetting.
j. The EP tube was put back on the magnetic grate and allowed to stand for 2 min. 48 μL of a supernatant was transferred to a new 1.5 mL EP tube and stored at −20° C. for later use.

4) Preparation of Sequencing Template

0.75 ng of a purified PCR product was used as a template, and subjected to water-in-oil PCR and enrichment of positive products to obtain a high-throughput DNA sequencing template. The reagents used were 3 components with small catalog numbers in the 510/520/530 TM Kit-Chef (A34019), including: Ion S5 chefsupplies (A27755), Ion chef solutions (A27754), and Ion 510/520/530chef regents (A34018). The experimental steps were as follows, and could be also referred to operating instructions of the 510/520/530 TM Kit-Chef (A34019).

Chef Water-In-Oil PCR and Chip Loading

a. Reagents and Consumables in Place

The Ion PGM TM Hi-Q TM View Chef Reagents and the Ion PGM TM Hi-Q TM Chef Solutions were added into 27756 and 27754 reagent chucks in the Ion 520/530Kit chef, respectively;
the upper right corner of the reagent chuck Ion PGM TM Hi-Q TM View Chef Reagents had 2 library holes for adding the sequencing library, namely the purified product obtained in step 2; and
consumables and chips were added.

b. Creation of Running Program

Log in to Server to Start Plan Creation
Plan→Templates→Whole Genome→Plan New Run→IonReporter→Application→Next
Parameter setting
Sample Preparation Kit: blank;
Library Kit Type: blank;
Template Kit: Ion 520&530Kit Chef
Templating Size: 400;
Sequencing Kit: Ion S5 Sequencing Kit;
Flow: 520 or 840;
Chip Type: 520chip;
Plugins: FileExporter;
Enter the project name and start running;
Projects→add a new “Project”→Plan→fill in the experiment name to Run Plan Name.

5) High-Throughput DNA Sequencing

The chip after loading was added into an S5 or S5 XL equipment to start sequencing. The reagents used were 2 components with small catalog numbers in 510/520/530 TM Kit-Chef (A34019), including: Ion S5 sequencing solutions (A27767) and IonS5 sequencing regents (A27768). The experimental steps were as follows, and could be also referred to operating instructions of the 510/520/530 TM Kit-Chef (A34019).

The operation steps of the S5 equipment were as follows:
a. turn on the power switch;
b. touch the screen to initialize;
c. add the Ion S5 Wash solution bottle with the small catalog number 27767 in the Ion 520/530Kit chef;
d. add the Ion S5 cleaning solution bottle with the small catalog number 27767 in the Ion 520/530Kit chef;
e. empty a waste liquid tank;
f. add the Ion S5 sequencing regent cartridge with the small catalog number 27768 in the Ion 520/530Kit chef;
g. add the chip after loading in step 3;
h. after everything was ready, click Next to start.

6) Data Analysis

a. Data Quality Control Results

The amount of data obtained by different chips was different. Taking one of the 520 chips as an example, the results were shown in FIG. 3. The effective area in the chip was 94%, of which 99% was linked with the library. 28.4% polyclonals, 11.9% low-quality libraries and 0% test fragment were removed; the final effective library was 59.7%, with a total of 6.9×10⁶ reads; an average read length of the fragments was 310 bp, yielding a total of 2.13 Gb of raw sequencing data.

b. Classification of Sequencing Information

According to the sample tag sequence information, the sequencing data were classified into different sample folders, and the results were as follows:

TABLE 6
Sample tag classification results

| | Number of | Sample tag | Number of | Sample tag | Number of | | Number of |
|---|---|---|---|---|---|---|---|
| Sample tag No. | sequencing | No. | sequencing | No. | sequencing | Sample tag No. | sequencing |
| barcode001 | 3381530 | barcode011 | 52755 | barcode021 | 64071 | barcode031 | 63318 |
| barcode002 | 150873 | barcode012 | 61214 | barcode022 | 257648 | barcode032 | 68201 |
| barcode003 | 51281 | barcode013 | 47439 | barcode023 | 91980 | barcode033 | 58947 |
| barcode004 | 142962 | barcode014 | 33061 | barcode024 | 78880 | barcode034 | 29074 |
| barcode005 | 134333 | barcode015 | 42572 | barcode025 | 102635 | barcode035 | 44606 |
| barcode006 | 105587 | barcode016 | 74482 | barcode026 | 71364 | barcode036 | 32848 |
| barcode007 | 60085 | barcode017 | 31497 | barcode027 | 57655 | barcode037 | 70967 |
| barcode008 | 108503 | barcode018 | 48408 | barcode028 | 39988 | barcode038 | 39734 |
| barcode009 | 71192 | barcode019 | 22828 | barcode029 | 43889 | barcode039 | 48045 |
| barcode010 | 176430 | barcode020 | 42900 | barcode030 | 45132 | barcode040 | 101478 |

| | |
|---|---|
| Others (unclassified due to sequencing errors) | 298574 |
| Total | 6548939 |

c. Identification of Sequence Microvariations

There were 937 Mb data matching to the reference sequence, accounting for 44% of the total sequence number, with an average sequencing depth of 1500×, where the amount of data that reached 99% match to the reference sequence was 851 Mb. Taking a mutation ratio ≥80% and the number of sequencing reads ≥50 as the screening criteria, the sequence microvariations were screened in the mitochondrial genome. Excluding barcode001 as a quality control material 2800, the distribution of mutation sites in the other 39 blood slice samples was shown in Table 7.

TABLE 7
Distribution of number of point mutations
in 39 blood slice samples

| | Number of |
|---|---|
| Sample No. | point mutations |
| 1 | 33 |
| 2 | 34 |
| 3 | 10 |
| 4 | 9 |
| 5 | 9 |
| 6 | 15 |
| 7 | 15 |
| 8 | 33 |
| 9 | 26 |
| 10 | 27 |
| 11 | 15 |
| 12 | 10 |
| 13 | 9 |
| 14 | 15 |
| 15 | 9 |
| 16 | 26 |
| 17 | 26 |
| 18 | 14 |
| 19 | 22 |
| 20 | 6 |
| 21 | 34 |
| 22 | 33 |
| 23 | 10 |
| 24 | 23 |
| 25 | 22 |
| 26 | 21 |
| 27 | 10 |
| 28 | 34 |
| 29 | 34 |
| 30 | 33 |
| 31 | 35 |
| 32 | 40 |
| 33 | 29 |
| 34 | 40 |
| 35 | 39 |
| 36 | 9 |
| 37 | 10 |
| 38 | 10 |
| 39 | 39 |

Taking a sample 1 as an example, compared with the reference sequence (NC_012920.1 Cambridge Revised Human Mitochondrial Whole Genome Sequence), the sample had a total of 33 point mutations in bases No. 73, 150, 489, 1048, 1107, 1438, 2706, 4048, 4769, 4883, 5153, 5178, 5301, 7028, 8701, 8857, 8860, 9180, 9540, 9667, 10397, 10398, 10400, 11176, 11719, 12705, 15043, 15301, 15326, 15724, 16223, 16362, 16519 of the mitochondrial genome (Table 8). Among them, 6 occurred in the mitochondrial hypervariable regions (hypervariable region 1: 16024 to 16569, hypervariable region 2: 1 to 576), and the remaining 27 occurred in the coding region. Compared with the mitochondrial sequencing method limited to hypervariable regions, the HTS kit of human mitochondrial whole genome (mtDNA) by fusion primer direct expansion improved a polymorphism information content, reduced a chance of random matching of personnel, and improved an ability of mitochondrial maternal lineage screening.

TABLE 8
Single base mutations in mitochondrial whole genome sequencing of sample 1

| | | Reference | | Mutated | | |
|---|---|---|---|---|---|---|
| Position | Depth | sequence | Proportion | sequence | Proportion | Description |
| 73 | 2363 | A | 0.5% | G | 99.5% | intergenetic region |
| 150 | 2360 | C | 1.5% | T | 98.5% | intergenetic region |
| 489 | 266 | T | 0.4% | C | 99.6% | intergenetic region |
| 1048 | 1802 | C | 9.4% | T | 90.6% | gene = RNR1; gene_biotype = rRNA |
| 1107 | 1777 | T | 9.5% | C | 90.5% | gene = RNR1; gene_biotype = rRNA |
| 1438 | 4081 | A | 8.3% | G | 91.7% | gene = RNR1; gene_biotype = rRNA |
| 2706 | 1324 | A | 0.6% | G | 99.4% | gene = RNR2; gene_biotype = rRNA |
| 4048 | 2610 | G | 0.3% | A | 99.7% | gene = ND1; gene_biotype = protein_coding |
| 4769 | 1172 | A | 0.2% | G | 99.8% | gene = ND2; gene_biotype = protein_coding |
| 4883 | 973 | C | 9.7% | T | 90.3% | gene = ND2; gene_biotype = protein_coding |
| 5153 | 17556 | A | 8.9% | G | 91.1% | gene = ND2; gene_biotype = protein_coding |
| 5178 | 17456 | C | 8.8% | A | 91.2% | gene = ND2; gene_biotype = protein_coding |
| 5301 | 14400 | A | 10.8% | G | 89.2% | gene = ND2; gene_biotype = protein_coding |
| 7028 | 2315 | C | 9.5% | T | 90.5% | gene = COX1; gene_biotype = protein_c |
| 8701 | 1918 | A | 0.1% | G | 99.9% | gene = ATP6; gene_biotype = protein_c |
| 8857 | 1848 | G | 7.1% | A | 92.9% | gene = ATP6; gene_biotype = protein_c |
| 8860 | 1846 | A | 0.1% | G | 99.9% | gene = ATP6; gene_biotype = protein_c |
| 9180 | 6204 | A | 1.0% | G | 99.0% | gene = ATP6; gene_biotype = protein_c |
| 9540 | 2588 | T | 0.4% | C | 99.6% | gene = COX3; gene_biotype = protein_c |
| 9667 | 3021 | A | 0.1% | G | 99.9% | gene = COX3; gene_biotype = protein_c |
| 10397 | 2733 | A | 0.0% | G | 100.0% | gene = ND3; gene_biotype = protein_coding |
| 10398 | 2750 | A | 0.3% | G | 99.7% | gene = ND3; gene_biotype = protein_coding |
| 10400 | 2751 | C | 0.4% | T | 99.6% | gene = ND3; gene_biotype = protein_coding |
| 11176 | 2203 | G | 0.5% | A | 99.5% | gene = ND4; gene_biotype = protein_coding |
| 11719 | 9626 | G | 1.0% | A | 99.0% | gene = ND4; gene_biotype = protein_coding |
| 12705 | 2307 | C | 0.5% | T | 99.5% | gene = ND5; gene_biotype = protein_coding |
| 15043 | 2813 | G | 6.0% | A | 94.0% | gene = CYTB; gene_biotype = protein_coding |
| 15301 | 2424 | G | 5.0% | A | 95.0% | gene = CYTB; gene_biotype = protein_coding |
| 15326 | 2435 | A | 0.2% | G | 99.8% | gene = CYTB; gene_biotype = protein_coding |
| 15724 | 1362 | A | 6.0% | G | 94.0% | gene = CYTB; gene_biotype = protein_coding |
| 16223 | 2176 | C | 15.2% | T | 84.8% | intergenetic region |
| 16362 | 5110 | T | 1.9% | C | 98.1% | intergenetic region |
| 16519 | 5178 | T | 0.4% | C | 99.6% | intergenetic region |

Therefore, the kit for HTS of human mitochondrial genome by direct amplification with fusion primer has a low detection cost and a convenient operation.

Finally, it should be noted that the foregoing embodiments are only intended to describe, rather than to limit the technical solutions of the present invention. Although the present disclosure is described in detail with reference to the preferred embodiments, a person of ordinary skill in the art should understand that modifications or equivalent replacements may be made to the technical solutions of the present disclosure without departing from the spirit and scope of the technical solutions of the present disclosure.

1. A fusion primer, comprising:
an upstream fusion primer and a downstream fusion primer, wherein the upstream fusion primer is sequentially ligated in series with an adapter A, a sample tag, and a target-fragment upstream primer from a 5′-end to a 3′-end;
wherein the downstream fusion primer is sequentially ligated in series with an adapter P and a target-fragment downstream primer from a 5′-end to a 3′-end;
wherein the adapter A is a sequencing adapter for sequencing with a universal primer, and the adapter P is a captured particle binding region for binding a captured magnetic bead; and
wherein the target-fragment upstream primer and the target-fragment downstream primer configured to amplify a target fragment of a human mitochondrial whole genome.

2-12. (canceled)

13. The fusion primer according to claim 1, wherein the adapter A has a length of 30 bp; the sample tag has a length of 10 bp; the target-fragment upstream primer has a length of 19 bp to 23 bp; the adapter P has a length of 23 bp; and the target-fragment downstream primer has a length of 19 bp to 23 bp.

14. The fusion primer according to claim 1, wherein the adapter A has a nucleotide sequence shown in SEQ ID NO: 89; and the adapter P has a nucleotide sequence shown in SEQ ID NO: 90.

15. A multiplex PCR primer pool, comprising the fusion primer according to claim 1 tagged with different sample tags.

16. The multiplex PCR primer pool according to claim 15, wherein the fusion primer comprises target fragment-specific primers shown in any one of SEQ ID NO: 1 to SEQ ID NO: 88;
wherein the target fragment-specific primers shown in SEQ ID NO: 1 to SEQ ID NO: 44 are upstream primers, and the target fragment-specific primers shown in SEQ ID NO: 45 to SEQ ID NO: 88 are downstream primers;
wherein the upstream primer shown in SEQ ID NO: 1 corresponds to the downstream primer shown in SEQ ID NO: 45, and the upstream primer shown in SEQ ID NO: 2 corresponds to the downstream primer shown in SEQ ID NO: 46, and so on.

17. The multiplex PCR primer pool according to claim 16, wherein 44 pairs of the fusion primers in each primer pool have concentrations shown in the following table:

| Primer name (indicated by a position of | Concentration (M/PCR |
|---|---|
| a product in a mitochondrial genome) | reaction system) |
| 17-332 | 0.01-0.03 |
| 361-774 | 0.05-0.07 |
| 869-1253 | 0.03-0.05 |
| 1347-1677 | 0.03-0.05 |
| 1697-2125 | 0.06-0.08 |
| 2167-2531 | 0.03-0.05 |
| 2549-2932 | 0.03-0.05 |
| 2986-3380 | 0.06-0.08 |
| 3395-3756 | 0.1-0.3 |
| 3790-4173 | 0.03-0.06 |
| 4187-4581 | 0.01-0.04 |
| 4639-5026 | 0.03-0.05 |
| 5045-5378 | 0.05-0.09 |
| 5382-5713 | 0.05-0.08 |
| 5718-6069 | 0.04-0.07 |
| 6070-6447 | 0.04-0.07 |
| 6458-6839 | 0.04-0.07 |
| 6859-7191 | 0.03-0.05 |
| 7238-7612 | 0.05-0.08 |
| 7617-7958 | 0.04-0.07 |
| 7992-8304 | 0.04-0.07 |
| 8333-8675 | 0.04-0.07 |
| 8676-8978 | 0.06-0.08 |
| 8982-9330 | 0.04-0.07 |
| 9339-9639 | 0.03-0.05 |
| 9640-9949 | 0.04-0.07 |
| 9973-10289 | 0.04-0.07 |
| 10294-10622 | 0.03-0.05 |
| 10651-10981 | 0.04-0.07 |
| 11002-11423 | 0.04-0.07 |
| 11460-11793 | 0.1-0.3 |
| 11798-12107 | 0.05-0.08 |
| 12113-12470 | 0.050-0.08 |
| 12471-12806 | 0.05-0.08 |
| 12836-13229 | 0.08-0.12 |
| 13234-13587 | 0.060-0.08 |
| 13607-13957 | 0.1-0.1 |
| 13990-14388 | 0.08-0.12 |
| 14453-14807 | 0.08-0.12 |
| 14817-15166 | 0.04-0.07 |
| 15187-15537 | 0.04-0.07 |
| 15550-15941 | 0.08-0.12 |
| 15971-16294 | 0.04-0.07 |
| 16316-16 | 0.04-0.07 |

18. The multiplex PCR primer pool according to claim 15, wherein the sample tags comprise a nucleotide sequence of tcacgaata and nucleotide sequences shown in anyone of SEQ ID NO: 91 to SEQ ID NO: 129.

19. A library preparation kit, comprising the multiplex PCR primer pool according to claim 15.

20. The library preparation kit according to claim 19, further comprising a DNA extraction-free PCR amplification enzyme, a PCR reaction buffer, a 2800 control DNA, and a DNA purification magnetic bead.

21. A high-throughput sequencing (HTS) kit of a human mitochondrial whole genome based on direct amplification of a fusion primer, comprising the library preparation kit according to claim 19, a sequencing template preparation kit, and a sequencing kit.

22. The kit for HTS of human mitochondrial genome by direct amplification with fusion primer according to claim 21, wherein the sequencing template preparation kit is purchased from Thermo Fisher Scientific.

23. The kit for HTS of human mitochondrial genome by direct amplification with fusion primer according to claim 21, wherein the sequencing kit is purchased from Thermo Fisher Scientific.

24. The multiplex PCR primer pool according to claim 15, wherein the adapter A has a length of 30 bp; the sample tag has a length of 10 bp; the target-fragment upstream primer has a length of 19 bp to 23 bp; the adapter P has a length of 23 bp; and the target-fragment downstream primer has a length of 19 bp to 23 bp.

25. The multiplex PCR primer pool according to claim 15, wherein the adapter A has a nucleotide sequence shown in SEQ ID NO: 89; and the adapter P has a nucleotide sequence shown in SEQ ID NO: 90.

26. The library preparation kit according to claim 19, wherein the fusion primer comprises target fragment-specific primers shown in anyone of SEQ ID NO: 1 to SEQ ID NO: 88; wherein the target fragment-specific primers shown in SEQ ID NO: 1 to SEQ ID NO: 44 are upstream primers, and the target fragment-specific primers shown in SEQ ID NO: 45 to SEQ ID NO: 88 are downstream primers;
Wherein the upstream primer shown in SEQ ID NO: 1 corresponds to the downstream primer shown in SEQ ID NO: 45, and the upstream primer shown in SEQ ID NO: 2 corresponds to the downstream primer shown in SEQ ID NO: 46, and so on.

27. The library preparation kit according to claim 26, wherein 44 pairs of the fusion primers in each primer pool have concentrations shown in the following table:

| Primer name (indicated by a position of | Concentration (M/PCR |
|---|---|
| a product in a mitochondrial genome) | reaction system) |
| 17-332 | 0.01-0.03 |
| 361-774 | 0.05-0.07 |
| 869-1253 | 0.03-0.05 |
| 1347-1677 | 0.03-0.05 |
| 1697-2125 | 0.06-0.08 |
| 2167-2531 | 0.03-0.05 |
| 2549-2932 | 0.03-0.05 |
| 2986-3380 | 0.06-0.08 |
| 3395-3756 | 0.1-0.3 |
| 3790-4173 | 0.03-0.06 |
| 4187-4581 | 0.01-0.04 |
| 4639-5026 | 0.03-0.05 |
| 5045-5378 | 0.05-0.09 |
| 5382-5713 | 0.05-0.08 |
| 5718-6069 | 0.04-0.07 |
| 6070-6447 | 0.04-0.07 |
| 6458-6839 | 0.04-0.07 |
| 6859-7191 | 0.03-0.05 |
| 7238-7612 | 0.05-0.08 |
| 7617-7958 | 0.04-0.07 |
| 7992-8304 | 0.04-0.07 |
| 8333-8675 | 0.04-0.07 |
| 8676-8978 | 0.06-0.08 |
| 8982-9330 | 0.04-0.07 |
| 9339-9639 | 0.03-0.05 |
| 9640-9949 | 0.04-0.07 |
| 9973-10289 | 0.04-0.07 |
| 10294-10622 | 0.03-0.05 |
| 10651-10981 | 0.04-0.07 |
| 11002-11423 | 0.04-0.07 |
| 11460-11793 | 0.1-0.3 |
| 11798-12107 | 0.05-0.08 |
| 12113-12470 | 0.050-0.08 |
| 12471-12806 | 0.05-0.08 |
| 12836-13229 | 0.08-0.12 |
| 13234-13587 | 0.060-0.08 |
| 13607-13957 | 0.1-0.1 |
| 13990-14388 | 0.08-0.12 |
| 14453-14807 | 0.08-0.12 |
| 14817-15166 | 0.04-0.07 |
| 15187-15537 | 0.04-0.07 |
| 15550-15941 | 0.08-0.12 |
| 15971-16294 | 0.04-0.07 |
| 16316-16 | 0.04-0.07 |

28. The library preparation kit according to claim 19, wherein the sample tags comprise a nucleotide sequence of tcacgaata and nucleotide sequences shown in anyone of SEQ ID NO: 91 to SEQ ID NO: 129.

29. The kit for HTS of human mitochondrial genome by direct amplification with fusion primer according to claim 21, further comprising a DNA extraction-free PCR amplification enzyme, a PCR reaction buffer, a 2800 control DNA, and a DNA purification magnetic bead.
